(19) United States
(12) Patent Application Publication
(10) Pub. No.: US 2009/0227843 A1
(43) Pub. Date: Sep. 10, 2009

(54) MULTI-INSTRUMENT ACCESS DEVICES AND SYSTEMS

(76) Inventors: Jeffrey A. Smith, Petaluma, CA (US); Geoffrey A. Orth, Sebastopol, CA (US); Michael S. Williams, Santa Rosa, CA (US); William L. Athas, Chapel Hill, NC (US); Daniel W. Fifer, Windsor, CA (US)

Correspondence Address: STALLMAN & POLLOCK LLP, P.O. Box 26450, SAN FRANCISCO, CA, 94126, US

(21) Appl. No.: 12/209,408

(22) Filed: Sep. 12, 2008

Related U.S. Application Data
(60) Provisional application No. 60/971,903, filed on Sep. 12, 2007.

(51) Int. Cl. A61B 1/32

(52) U.S. Cl. 600/208; 600/201

(57) ABSTRACT

A multi-instrument access device includes a base positionable within an opening (e.g. an incision or puncture) formed in a body wall and a dome-shaped seal on the base and positioned such that it is disposed outside the body wall during use. A plurality of instrument ports extend proximally from the seal for receiving instruments to be inserted into the body for use in a procedure. Tubular instrument tubes having pre-curved distal ends may be insertable through the ports for receiving the instruments and for orienting the operative ends of the instruments toward a target site.

### RELATED APPLICATIONS

This application claims the benefit of U.S. Provisional Application No. 60/971,903, filed Sep. 12, 2007, Attorney Docket No. TRX-1200, which is incorporated herein by reference.

### TECHNICAL FIELD OF THE INVENTION

The present invention relates to the field of access devices through which medical instruments may be introduced into an incision or puncture opening formed in a body wall.

### BACKGROUND

Surgery in the abdominal cavity is frequently performed using open laparoscopic procedures, in which multiple small incisions or ports are formed through the skin and underlying muscle and peritoneal tissue to gain access to the peritoneal site using the various instruments and scopes needed to complete the procedure. The peritoneal cavity is typically inflated using insufflation gas to expand the cavity, thus improving visualization and working space. Further developments have lead to systems allowing procedures to be performed using only a single port.

In single port surgery (“SPS”) procedures, it is useful to position a device within the incision to give sealed access to the operative space without loss of insufflation pressure. Ideally, such a device is partitioned in some manner to provide sealed access for multiple instruments. The present application describes a multi-instrument access device suitable for use in SPS procedures, and other laparoscopic procedures.

### BRIEF DESCRIPTION OF THE DRAWINGS

FIG. 1A is a perspective view of a first embodiment of an access device.

FIG. 1B schematically illustrates positioning of the access device of FIG. 1A through an incision in an abdominal wall.

FIG. 2 is an exploded perspective view of a second embodiment of an access device.

FIG. 3A is a top perspective view of the base of the access device of FIG. 2.

FIG. 3B is a side elevation view of the base of FIG. 3A.

FIG. 3C is a perspective view of the access device of FIG. 2 following coupling of the seal to the base.

FIG. 3D is a side elevation view showing the base and seal in assembled form.

FIG. 3E is a section view taken along the plane designated 3E-3E in FIG. 3D.

FIG. 4 is a bottom plan view of the base of FIG. 3A.

FIGS. 5A and 5B are elevation views of the seal of the first or second embodiment, showing one of the ports in a neutral position and in a pivoted position, respectively.

FIG. 6 is a cross-sectional perspective view of the seal of FIG. 5 with an adaptor/reducer stored on one of the ports.

FIG. 7 is a cross-sectional perspective view of the reducer of FIG. 6.

FIGS. 8A and 8B are perspective views illustrating attachment of detachable ports to a seal in an alternative embodiment.

FIG. 9A is a plan view of the port of FIG. 8A.

FIG. 9B is a side elevation view of the port of FIG. 9A.

FIG. 9C is a cross-section view taken along the plane designated 9C-9C in FIG. 9B.

FIG. 9D is a cross-section view similar to FIG. 9C showing a second, larger diameter, port.

FIG. 10 is a perspective view of the seal of FIGS. 8A and 8B with all ports attached.

FIG. 11 is a cross-section view taken along the plane designated 11-11 in FIG. 10.

FIG. 12 is a perspective view illustrating attachment of a detachable valve to the port of an alternative embodiment of a seal.

FIG. 13 is a perspective view of the seal of FIG. 12 with all valves attached.

FIG. 14 is a cross-section view taken along the plane designated 14-14 in FIG. 13.

FIG. 15 illustrates a system of instrument tubes that may be positioned in the access device.

FIG. 16 is a perspective view showing a distal portion of the access device positioned in an abdominal wall incision, with the instrument tubes extending through the access device into the abdominal cavity. Instruments are shown disposed in the instrument tubes.

FIG. 17 is a perspective view of an alternative access device.

FIG. 18 is a perspective view of the access device of FIG. 17 prior to insertion of the partitioning insert.

FIG. 19 is similar to FIG. 18 and further shows the locking screws;

FIG. 20 is a perspective view illustrating use of the FIG. 17 access device with right and left instrument tubes;

FIG. 21 is a perspective view similar to FIG. 21 and further showing use of the third instrument tube.

FIG. 22 is a perspective view of the access device of FIG. 17 using three instrument tubes.

### DETAILED DESCRIPTION

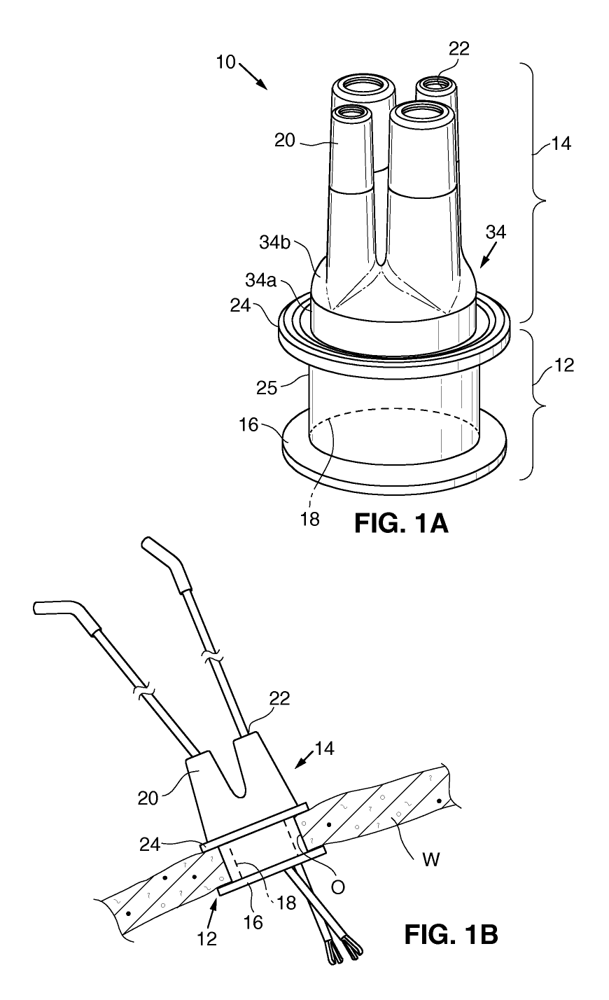

FIG. 1A illustrates a multi-instrument access device 10. The access device 10 includes a base 12 positionable within an opening (e.g. an incision or puncture) formed in a body wall, and a seal 14 on the base 12 and positioned such that it is disposed outside the body wall during use. In the first embodiment shown in FIG. 1A, the seal and base are integrally formed (e.g. molded into a single piece) or permanently attached from separately formed pieces.

Base 12 is a generally hollow or tubular member having a wall 25 defining a lumen 18 and a distal flange 16 surrounding the distal opening of the lumen. The flange and distal opening may be circular, elliptical, or any other shape suitable for insertion into an opening in the body wall. The base 12 is preferably constructed of a flexible material that allows the base 12 to be pinched or flattened into a smaller profile for insertion through the opening in the body wall, and that will preferably restore the base to its original shape and size after compression is released.

Flange 16 has a width that will define a sufficient margin around the border of the opening in the abdominal wall to prevent its inadvertent withdrawal from the opening during use. Although flange 16 is shown as a fully circumferential member, alternate elements that are not fully circumferential (e.g. two or more flange segments), may alternatively be used to perform the same retention function. By including a broad flange, the base is able to retract peritoneal tissue away from the base port, keeping the tissue from obstructing access and preventing tools and/or implants from inadvertently slipping between the abdominal wall and the peritoneal tissue.

Seal 14 includes a plurality of ports 20 extending proximally from the base 12. The ports 20 are tubular elements having proximal openings 22 for receiving medical instruments. The multiple ports 20 may be formed with equal diameters, or they may have different diameters as shown. For example, some of the ports may have 12 mm diameter openings, while others have 9 mm diameter openings. In the illustrated embodiments, each port is parallel to the other ports and is oriented such that its longitudinal axis is parallel to the longitudinal axes of the seal and the other ports. With this arrangement, the ports generally extend perpendicular to the tissue surrounding the incision.

As will be discussed in greater detail below, valves (not shown in FIG. 1A) are positioned within the ports 20 so as to maintain insufflation pressure within the abdominal cavity during use of the access device 10.

A proximal flange 24 (or equivalent structure) is positioned to contact the skin surrounding the opening in the abdominal wall, to prevent the access device from inadvertently being pushed into the body cavity during use.

During use, the flexible base 12 is folded or pinched and inserted into the opening O in the abdominal wall W and advanced until distal flange 16 is disposed beneath the abdominal wall W, with and proximal flange 24 remaining outside the body as shown in FIG. 1B. The base 12 is allowed to unfold such that the wall surrounding the base contacts the edges of the opening O, keeping the opening open for access by instruments. Instruments I are inserted through proximal openings 22 in the ports 20, through the lumen 18 in the base 12, and into the abdominal cavity. Flanges 16, 24 help to retain the base within the opening O without the need for suturing the base to the surrounding tissue, thereby improving cosmesis by eliminating scarring from the sutures and allowing the base to be rotationally repositioned within the incision if required during surgery. The shapes of the flanges 16, 24 and the wall surrounding lumen 18 of the base may be reinforced using resilient rings or other materials embedded in the polymeric material, or by thickened regions of the base material.

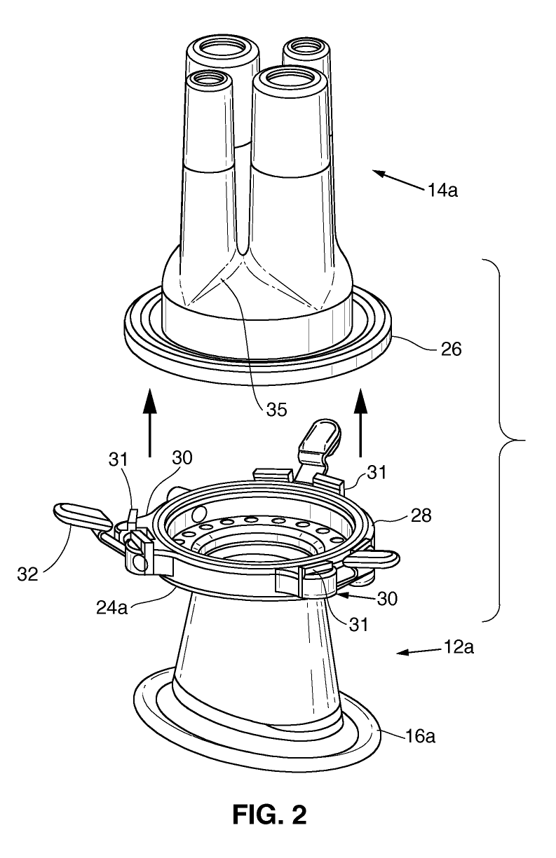

Suitable materials for the base 12 and/or seal 14 include thin walled or highly flexible polymeric materials including but not limited to silicone, urethane and carbothane. The configuration of flexible materials allows for maximum tool range of motion with minimal trauma to the tissue. For example, manipulation of tools used in the access device 10 might stretch one or more areas of the device (e.g. if tool handles are spread in opposite directions to bring the operative ends of instruments closer together). Due to the mechanical properties of the disclosed materials, the device 10 and surrounding tissue will stretch together, rather than forcing the access device to pop from the incision or driving a rigid port traumatically against the surrounding tissue. The ports 20 may include a lubricious lining to facilitate advancement of instruments through them. Some of the materials (e.g. in the base 12) may be loaded with anti-microbial agents such as silver nitrate. FIG. 2 shows an alternate embodiment in which the base 12a and seal 14a are separate pieces attachable to each other during use. In this embodiment, the seal 14a includes a first engaging portion which in this embodiment takes the form of a flange 26. The base 12a includes a second engaging portion positioned to engage the first engaging portion. In the illustrated embodiment, the second engaging portion includes a ring 28 on the base 12a. The flange 26 of the seal 14a seats against and makes sealing contact with the ring 28. The base includes three radially extending tabs 30, each of which includes guide elements 31 that extend upwardly from the tabs 30. Guide elements 31 help to center the flange 26 into the proper position as it is being lowered onto the base.

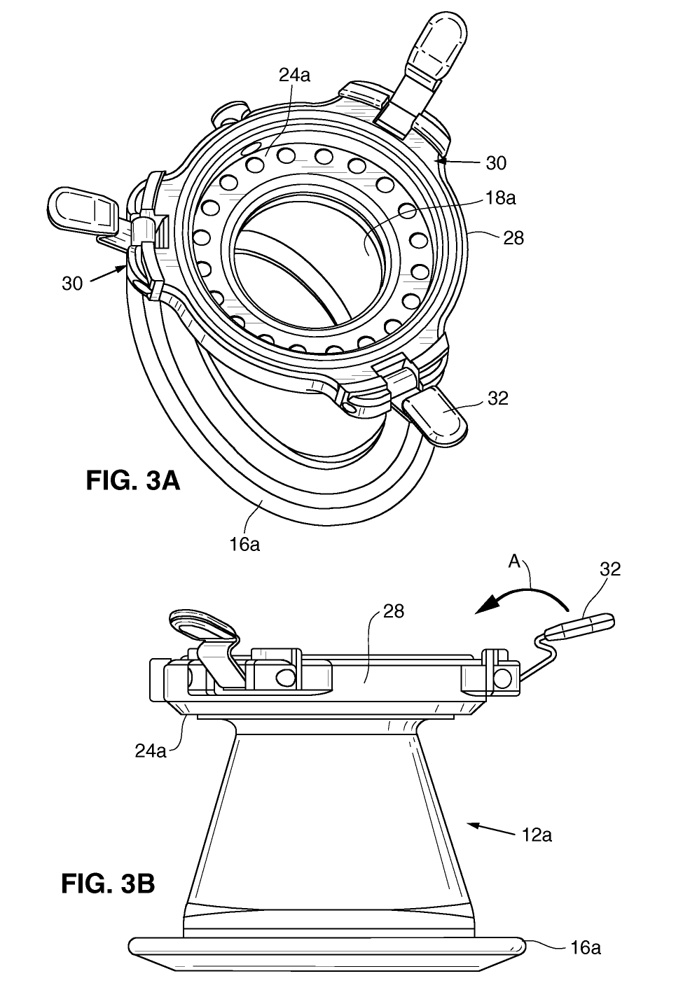

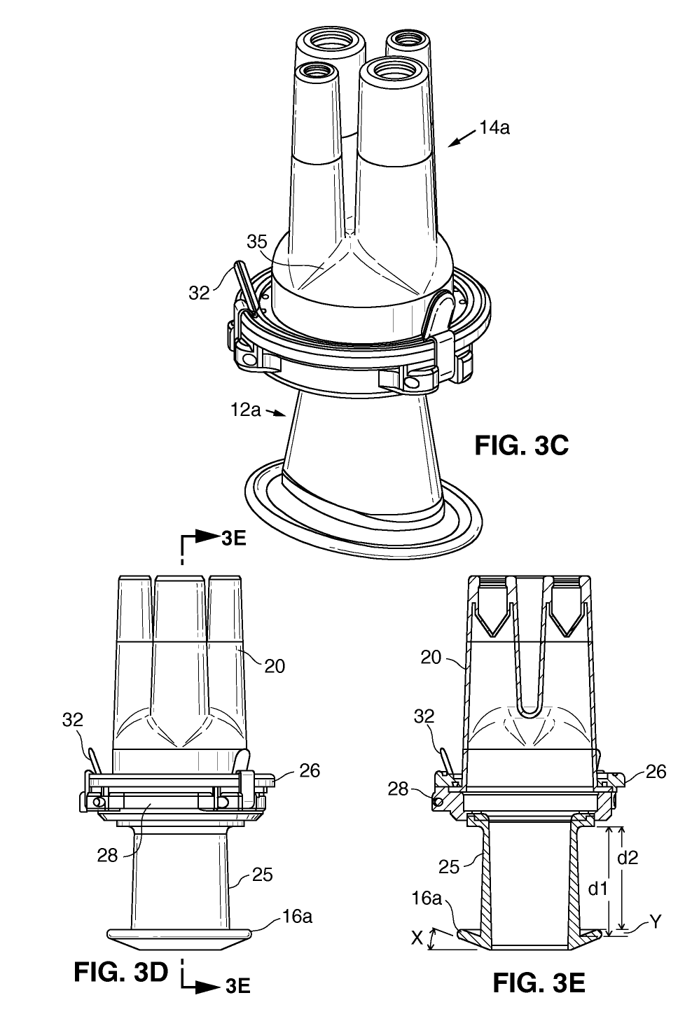

Clips 32 (preferably two or more) on the ring 28 are used to secure the base 12a to the seal 14a. The clips have an unclipped position shown in FIG. 3B and are inwardly pivotable in the direction of arrow A in FIG. 3B. Once the seal 14a is seated against the ring 28 on the base 12a, the clips are pivoted in this manner to a clipped position as shown in FIG. 3C. When in the clipped position, the clips 32 engage the inner circumference of flange 26 of the seal 14a, thus coupling the seal to the base. In the illustrated embodiment, the clips 32 are pivotally coupled to corresponding tabs 30 as shown. The ring 28 and flange 26 may be made of material that is stiffer than the material used for the other portions of the base, seal and ports (e.g. Shore D 80 for the ring and flange vs. Shore A 50 for the wall 34, ports 20 and base wall 25.

With this clip arrangement, the rotational position of the seal 14a relative to the base 12a is not critical. Any rotational position can be used, and the rotational position may be changed if necessary during a procedure. In alternative embodiments, an engaging portion of the base may be match to a specific engaging portion of the seal, thus requiring that the two be rotationally aligned.

Base 12 includes a wall 25 that may by cylindrical (FIGS. 3D and 3E) or that may have an inward or outward taper from the proximal to the distal end. In preferred embodiments, the interior surface of the wall 25 surrounding the base 12a has zero or minimal inward taper from the proximal end of the wall to the distal end of the wall. Tapers of less than approximately 5°, and preferably approximately 2-3° are preferred. This very slight taper facilitates removal of the base from the incision, while allowing for optimum range of motion for instruments extending from the ports through the base during use.

During use of the second embodiment, the base 12a may be placed in the opening in the body wall before the seal 14a is coupled to the base. This is particularly beneficial where an initial step in the procedure may involve an instrument or implant that is too large for the ports 20a. For example, where the access device 10a is to be used to implant a lap band or a Swiss lap band of the type used to induce weight loss, the lap band may be dropped through the lumen 18a in the base 12a and into the operative space. Then, once the seal 14a has been coupled to the base 12a, the implant may be retrieved from within the operative space using an instrument passed through the seal 14a.

Referring to FIG. 3E, distal flange 16a may angle upwardly by an angle “X” relative to a plane parallel to the longitudinal axis of the base 12a. By angling the flange, a variety of abdominal wall thicknesses can be accommodated, since the distance “d1” between flange 16a and flange 24a at the most radially inward portion of the flange is smaller than the distance d2 between them at the most peripheral portion of the flange. Additionally, if the abdominal wall (or a portion of the abdominal wall) is thicker than d2, the flange 16a will pivot in response to the larger tissue thickness as indicated by arrow Y.

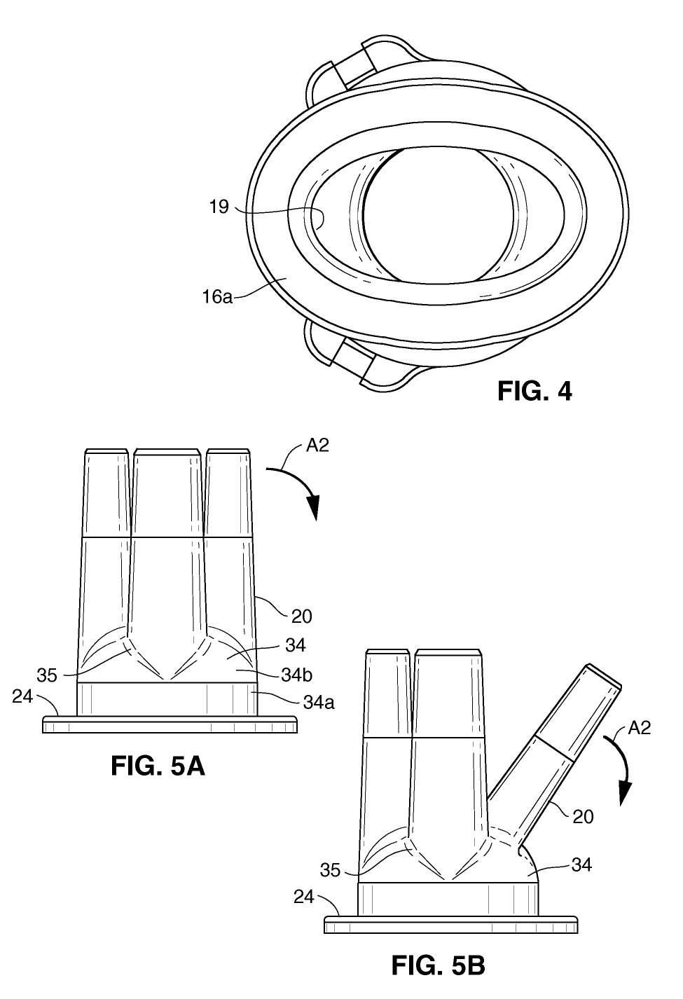

FIG. 4 illustrates that the distal flange 16a and distal opening 19 of the base 12a may have an elliptical shape. This configuration may be particularly convenient when the opening in the body wall is an elongate incision, or when thoracic access between ribs is required.

Features that may be included on the seals 14, 14a of the first and/or second embodiments will next be described. For simplicity, reference numbers matching those used to describe the first embodiment will be used in the following description.

Referring to FIG. 5A, the seal 14 may be molded to include a surface or wall 34 from which the ports 20 extend. The wall 34 (or a combination of walls or surfaces) is shaped so as to define a three dimensional volume of space within the seal proximal to the flange 16a yet distal to the distal openings of the ports 20. With this arrangement, the wall 34, and thus the distal opening of each port, is proximally offset from the incision rather than directly between the open edges of the wound. The seal 14 is constructed to allow the ports 20 to move somewhat relative to the wall 34 (e.g. to deflect or pivot relative to the wall 34 as indicated by arrow A2 in FIG. 5A) during use of tools positioned within those ports. Allowing the ports to move in response to instrument movement minimizes trauma to the incision by avoiding movement of the base within the incision when an instrument shaft is pivoted.

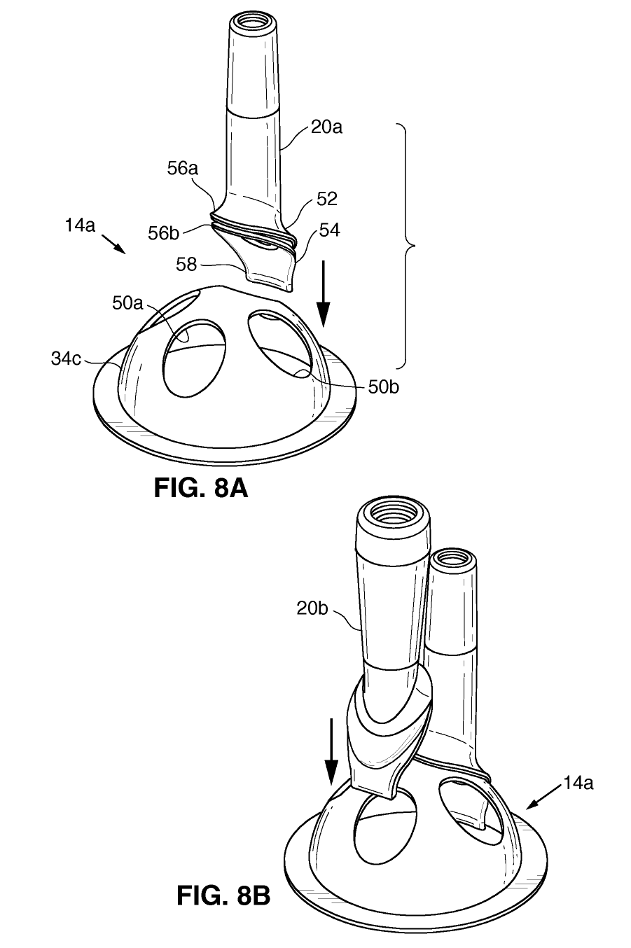

Additional range of motion may be given to the ports 20 by giving the wall 34 a contour, such as the dome shape shown in FIG. 5A and elsewhere. The dome shown in FIG. 5A includes a cylindrical lower portion 34a and an upper portion 34b that is continuously curved or that radiuses from the cylindrical portion to a relatively planar top surface. In other seals, such as the one that will be discussed in connection with FIGS. 8A and 8B, the domed wall 34 may have a continuous curvature. In the dome shaped embodiments, the dome may be partially spherical or it may have an alternative angle of curvature.

The ports 20 preferably extend from a curved portion of the wall 34 or dome. In some embodiments, the area of the seal where the wall of a port 20 meets the domed wall 34 includes a teardrop shaped band or junction 35. When an instrument disposed in a port 20 imparts forces against the port in a direction transverse to the longitudinal axis of the port, preferential bending along the junction occurs so as to prevent kinking of the port. When a port pivots radially as shown in FIG. 5C, the apex of the port may deflect the surrounding dome wall slightly inwardly. By causing the dome wall to deflect, deflection of the port wall is avoided, thus preventing the wall of the port constricting the port's lumen in the region of the junction. The junction 35 may be formed with a thinner and/or more flexible material to facilitate bending at the junction.

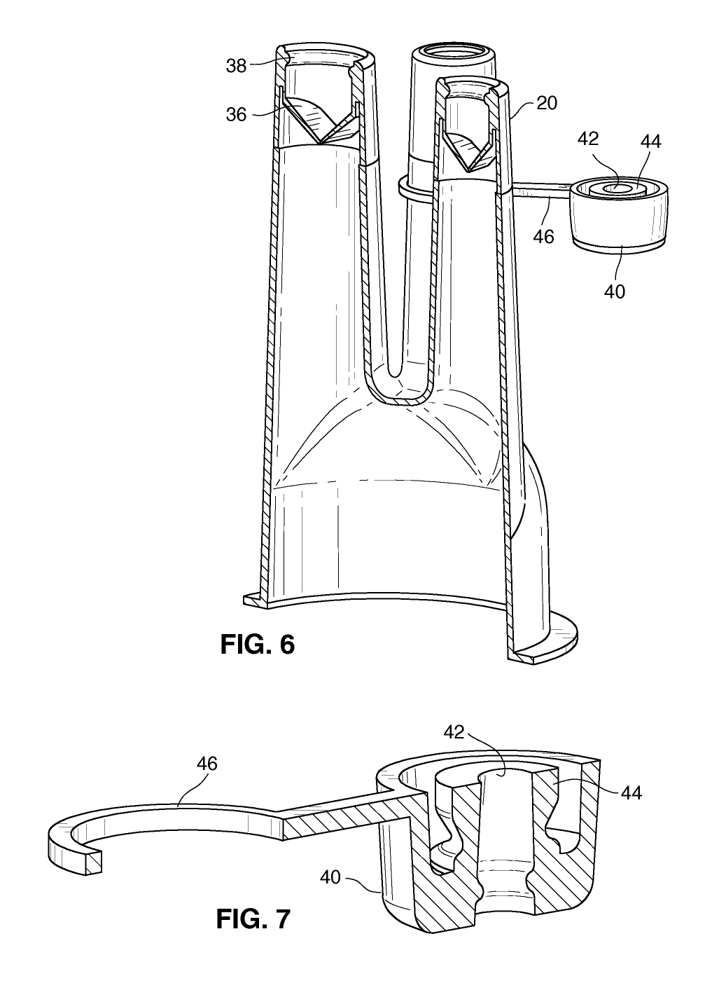

Referring to the cross-section view of FIG. 6, each port 20 is equipped with a sealing system having a first seal providing for self-sealing of the port in the absence of a medical instrument within the port, and a second seal that creates a seal against the shaft of instruments passed into the port. A preferred sealing system uses components that will not significantly increase the overall footprint of the corresponding port 20, so as to maximize the number of tool ports 20 available for a given incision size. In the FIG. 6 configuration, an annular seal 38 positioned at or near the proximal opening of the port 20, and a duck-bill valve 36 located distal to the annular seal 38. During use, duck-bill valve 36 remains closed when there is no instrument in the port 20. Instruments passed through the port 20 will pass between the flaps of the valve 36, thus releasing the seal provided by the valve 36. However, this will not result in appreciable loss of sealing, since insertion of the instrument into the port 20 causes the annular seal 38 to make sealing contact with the instrument shaft. In preferred embodiments, the ports 20, wall 24, and one or both of the valve 36 and/or seal 38 are formed as an integral piece by molding or other processes.

Different ones of the ports 20 may be provided to have proximal openings of various diameters to give access to a variety of tool sizes. Additionally, the seal 14 may include other features that allow use of a diverse range of tool sizes. Referring still to FIG. 6, seal 14 may include one or multiple adaptors 40 or port reducers attachable to the ports 20. Adaptors 40 can be provided in a number of sizes to allow various smaller diameter instruments to be used without compromising the ability of the port to seal against the smaller tools. Adaptor 40 may be a plug insertable into one of the ports 20 such that the outer surface of the adaptor makes sealing contact within the annular seal 38. Referring to the cross-section view of FIG. 7, within the adaptor 40 is a small diameter lumen 42 (e.g. 5 mm diameter) surrounded by an annular seal 44 that will seal against the shaft of a small diameter instrument. A mount 46 may be used to temporarily couple adaptor 40 to the seal 14 so it is readily available when needed during a procedure.

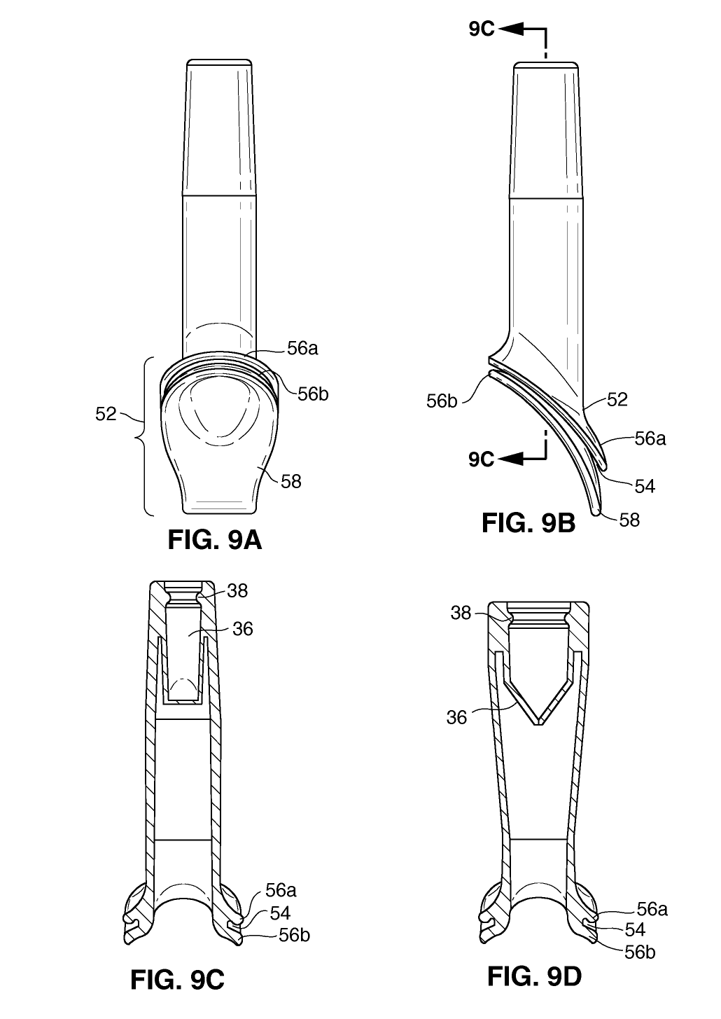

FIGS. 8A and 8B show an alternative seal 14a that may be used with the base 12 of FIG. 1. Seal 14a includes a wall 34c and openings 50a, 50b in the wall 34c and detachable ports 20a, 20b are insertable into the openings 50a, 50b. As shown in FIGS. 9A-9C, each port 20a is a tubular element including an internal duckbill valve 36 and annular instrument seal 38 similar to those described above in connection with the first embodiment. The ports may all be of equal size, or the sizes may differ between the ports. In the embodiment shown in FIGS. 8A through 11, two sizes of ports are used. For example, port 20b (FIGS. 8B and 9D) might have an opening proportioned to receive and seal against 10 mm instruments, whereas port 20a could have an opening proportioned to receive and seal against 5 or 7 mm instruments. In a preferred seal 14a, the ports are designed so that the openings 50a, 50b in the seal 14a are uniform in size, allowing ports of different sizes to be interchanged as needed.

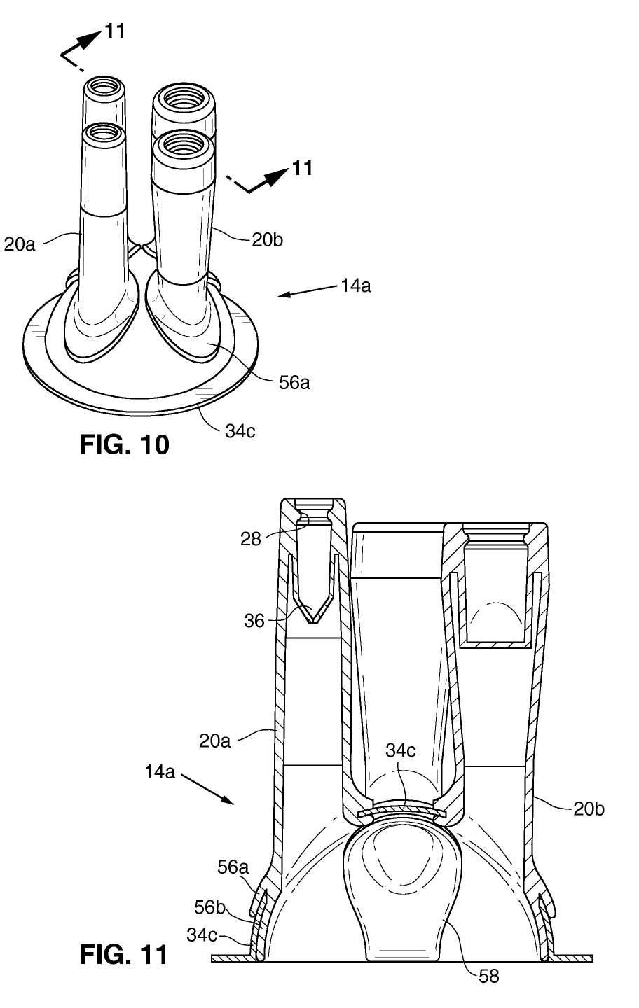

The distal end includes a transverse flange 52 having a circumferential groove 54 disposed between circumferential lips 56a, 56b. The distalmost one of the lips 56b includes a tongue 58 at its distal end. To mount the port 20a to the seal 14a, tongue 58 is inserted into opening 50a (FIG. 8A). The port 20a is pressed downwardly to cause lip 56b to seat below the edge of opening 50a and to cause lip 56a to contact the portion of the wall 34c surrounding the opening 50a on the exterior of the seal 14a, thereby forming a seal around the opening 50a. Also see FIGS. 10 and 11. The process is repeated for the remaining ports

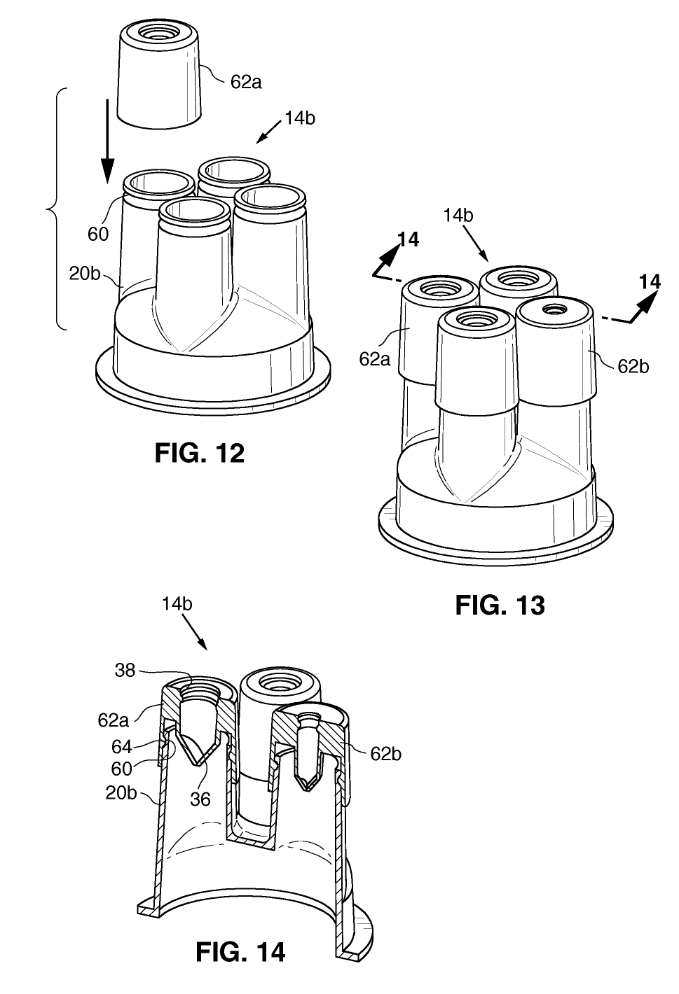

FIGS. 12-14 show yet another alternative seal 14b that may be used with the base 12. In this embodiment, seal 14b includes ports 20b that may be of uniform size as shown. Each port 20b includes an annular groove 60 adjacent its proximal opening. A plurality of valve caps 62a, 62b are provided for attachment to the ports 20b. A preferred system is provided with caps having openings of various sizes to accommodate instruments of differing shaft diameters.

As shown in FIG. 14, each valve cap has a sealing system having a first seal providing for self-sealing of the port in the absence of a medical instrument within the port, and a second seal that creates a seal against the shaft of instruments passed into the port. As with the earlier described embodiments, the preferred seals are a duckbill valve 36 and an instrument seal 38. The interior wall of the valve cap has a lip 64 positioned to seat within the groove 60 of a port 20b and to thereby seal the cap against the port.

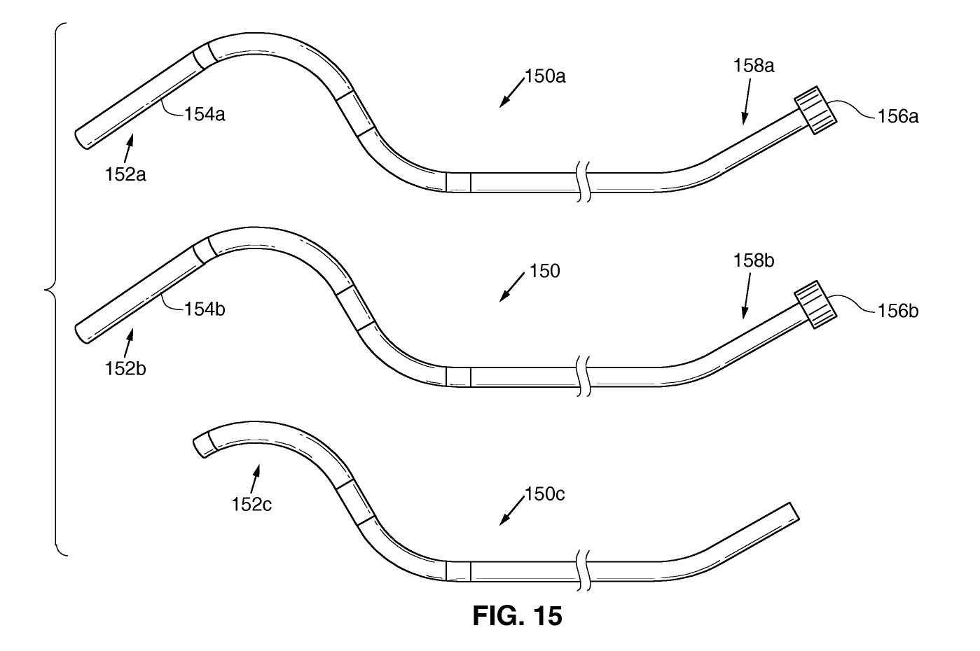

Although FIG. 1B shows instruments inserted directly into the access device 10, the access device may be used as part of system that includes instrument cannulas that are passed through the ports 20 in the access device and used to receive instruments. For example, referring to FIG. 15, the access device may be used as part of a system that includes multiple instrument tubes 150a, 150b, 150c that are placed in the ports of the access device (see ports 20 and device 10 in FIG. 1A). During use of such a system, an opening (e.g. incision or trocar puncture) is formed in an abdominal wall, and the access device (e.g. device 10 of FIG. 1A) is seated within the opening. One or more of the instrument tubes 150a-c is inserted into the abdominal cavity via the access device. Instruments needed for carrying out the necessary medical procedure are passed through insertion openings (not shown) at the proximal ends of the instrument tubes and put to use within the abdominal cavity.

Each instrument tube 150a-c is provided with a pre-shaped curve in its distal region 152a-c. The curve for each instrument tube is selected to orient that tube such that when it is disposed through access device positioned in a body wall incision, instruments passed through the lumen of the instrument tube can access a target treatment site. The various instrument tubes used with the system may all have the same size and/or geometry, or two or more different sizes and/or geometries may be used. The curve in any given instrument tube may be continuous or compound, and it can be formed to occupy a single plane or multiple planes.

In the illustrated example, each of tubes 150a and 150b has a deflectable region 154a-b that is deflectable in one or more directions to allow orientation of the distal openings of the tubes 150a-b to allow positioning and manipulation of the operative ends of the instruments disposed within the tubes 150a-b. This may avoid the need for sophisticated steerable surgical instruments and allows simple instruments having flexible shafts to be positioned in the tubes so that steering of the instruments is achieved by deflecting the tubes. Deflection of deflectable regions 154a-b is accomplished with pullwires or other means using methods known to those skilled in art. Pullwire actuators 156a, 156b are disposed on the proximal sections 158a, 158b of the tubes 150a, 150b (which remain outside the body throughout the procedure), and may include locking features allowing a user to lock the deflected position of a tube.

Any or all of the tubes may be constructed without a deflectable section, as is the case with tube 150c.

The proximal section 158a, 158b, 158c of each tube can likewise include a fixed curve. This feature causes the proximal ends to flare away from one another when the tubes are disposed in the ports, thus minimizing interference between the handles of instruments positioned in the tubes 150a-150c.

The tubes 150a-c may be formed of any material that will provide sufficient rigidity to prevent buckling during use. In one embodiment, tubes 150a, 150b have proximal portions formed of stainless steel or similarly rigid material, and deflectable regions 154a, 154b made using a flexible biocompatible polymeric material such as those currently used for medical catheters.

The interior lumen of the tubes 150a-c may be provided with sealing means (e.g. o-ring seals) to prevent loss of pressure between the instrument shafts and surrounding lumen walls.

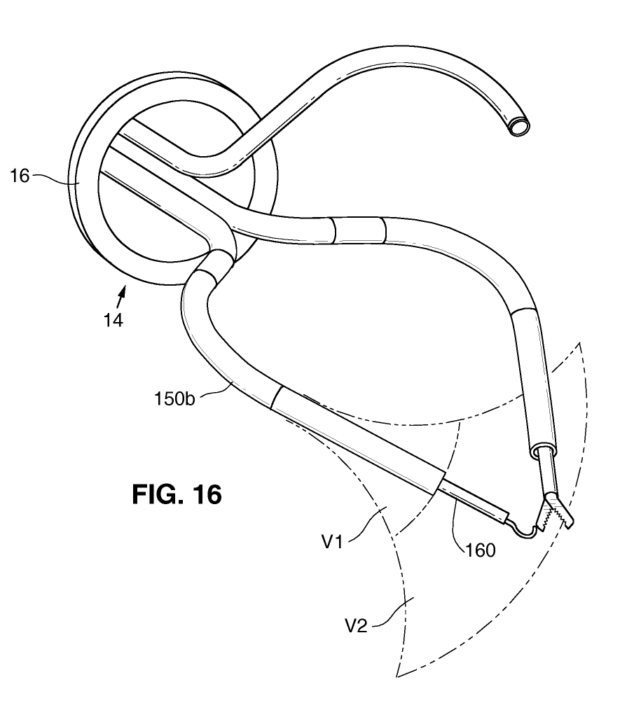

During use, each one of the instrument tubes 150a, 150b is passed through the access device by inserting its distal end into one of the ports 20 in the seal 14 (FIG. 1A). FIG. 16 shows the orientation of tubes 150a, 150b extending side by side into the abdominal cavity from a pair of the ports (the individual ports are not visible in FIG. 16). The tubes 150a, 150b may be rotated about their longitudinal axes to orient their distal openings towards a common operative site within the abdominal cavity. The proximal-to-distal positions of the tubes 150a, 150b may also be fine-tuned by sliding them inwardly or outwardly. Friction between each tube and the annular seals (e.g. annular seals 38 of FIG. 6) within its corresponding one of the ports 20 retains the longitudinal and rotational position of the tubes within the ports 20.

The surgeon will select an instrument needed to perform a procedure within the body cavity, and s/he will insert that instrument (see instruments 160, 162) into one of the tubes 150a, 150b. Additional instruments are selected and likewise advanced through the most suitable ones of the tubes. As instrument changes are made throughout the procedure, different combinations of the tubes 150a-c and/or ports 20 may be utilized. In some instances, one or more of the tubes 150a-150c may be used for some instruments, while other instruments may be inserted directly through one of the ports 20. Likewise, an endoscope may be positioned in one of the tubes, or directly into one of the ports 20.

As illustrated in FIG. 16, during the course of the procedure, the deflectable regions 154a, 154b of the tubes may be manipulated through the use of pullwire actuators 156a, 156b (FIG. 15) to change the orientation of the instruments within the tubes. The figure shows in dashed lines V1 a conical volumes defined by an exemplary movement pattern for the tube 150b, and the corresponding volume V2 defined by a tool 160 within the tube 150b.

Additionally, the tubes 150a, 150b and/or 150c may be rotated or longitudinally advanced/rotated as needed to reposition their corresponding instruments. Following the procedure, the instruments are removed from the tubes 150a-c, and the access device is removed from the body.

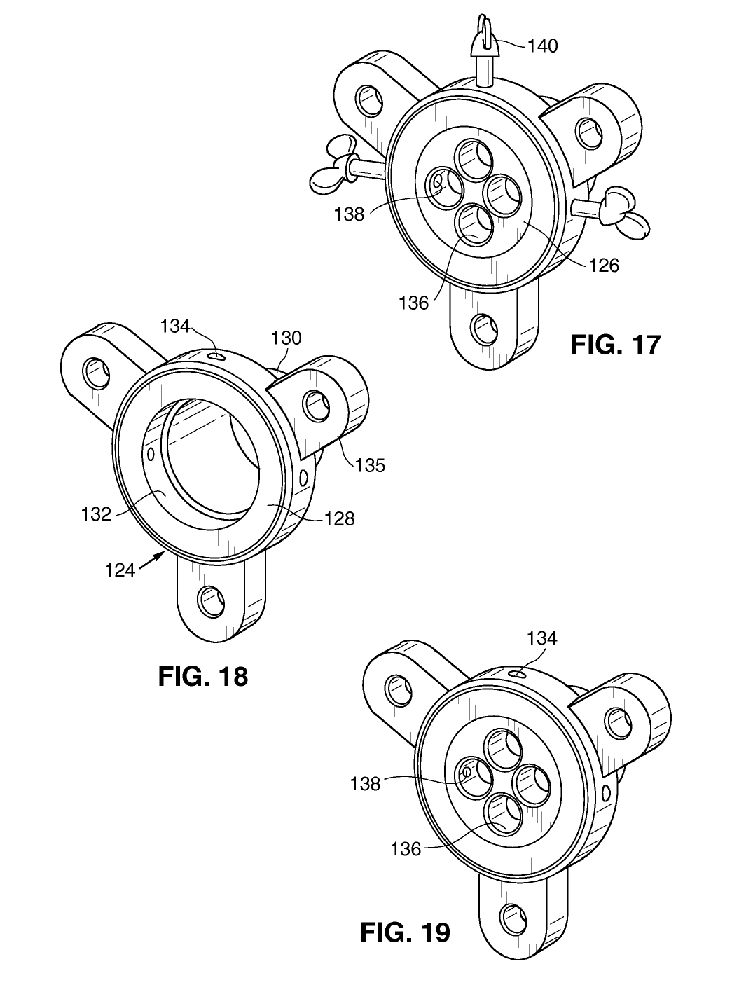

In an alternate system, the tubes 150a-c may be used with an alternate access device or port of the type shown in FIG. 17. The access port 114 includes a tubular port 124 and a partition insert 126. Details of the tubular port 124 are best seen in FIG. 18, which illustrates a collar 128 and a tube 130 extending proximally from the collar 128. The tube 130 preferably has a smaller outer diameter than the collar 128, allowing for positioning of the tube 130 within an incision while the collar 128 remains in contact with skin surrounding the incision. Insufflation gas used to inflate the abdominal cavity will expand the abdominal wall outwardly, facilitating formation of a seal between the collar and the tissue surrounding the incision. If necessary, a substance or material (e.g. silicone, rubber, adhesive, gel, etc.) may be positioned between the collar and the tissue to facilitate sealing

A large central bore 132 extends through the port 124. Throughbores 134 extend in a radial direction through the collar 128 as shown.

One or more flanges 135 extend radially outward from the collar 128. During use, these flanges may be coupled to a rail of the surgical table.

Referring to FIG. 19, partition insert 126 is a disk proportioned to be engaged within the proximal opening of the collar 128 as shown. The collar 128 and/or insert 126 may include materials or features allowing a seal to form around the perimeter of the insert 126 to prevent loss of insufflation pressure during use.

A plurality of openings 136 in the insert 126 provide individual entry points for the instrument tubes 150a-150c and/or for any instruments that can be advanced to the operative site without an instrument tube. A selection of inserts may be provided, each having a different combination of opening sizes and arrangements.

Threaded bores 138 in the insert 126 are positioned in alignment with throughbores 134 of the collar 128. Locking screws 140 (FIG. 17) are screwed into the throughbores 134 and corresponding threaded bores 138 of the insert such that, when tightened, they will contact with the shafts of instrument tubes 150a-150c extending through openings 136. This feature allows the tubes to be secured within the openings 136 in a desired orientation. Seals (e.g. o-rings) may be provided within the openings 136 to allow sealing around the instrument tubes.

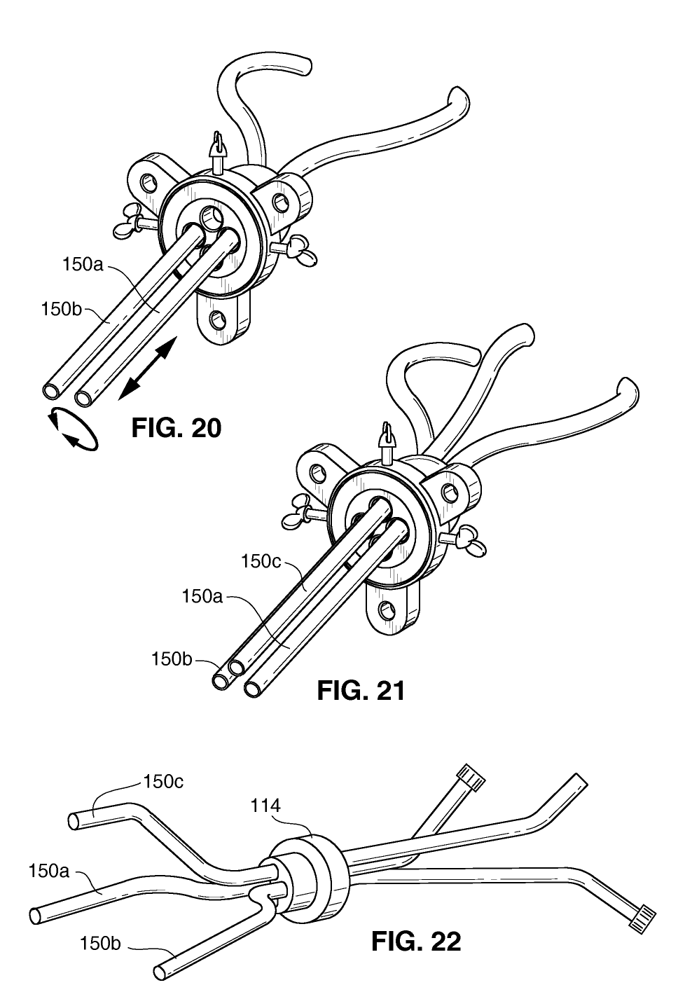

FIGS. 20 and 21 illustrate use of a system utilizing access port 114 and tubes 150a-c. According to one method of using the system 10, the port 124 is placed with the tube 130 (FIG. 13) extending into an opening formed in the abdominal wall. The partition insert 126 is secured within the tubular port 124 either before or after the port is positioned. Next, each one of the instrument tubes 150a, 150b is passed through the partition insert 126 by inserting its distal end into one of the openings 136 in the partition insert 126. FIG. 20 shows tubes 150a, 150b positioned in the left-most and right-most ones of the openings. The tubes 150a, 150b are rotated about their longitudinal axes to orient their distal openings towards a common operative site. The proximal-to-distal positions of the tubes 150a, 150b may also be fine-tuned by sliding then inwardly or outwardly. Finally, the screws 140 associated with the left and right openings of the partition insert are tightened against the shafts of the tubes 150a, 150b to set their respective positions.

In the illustrated method the third tube 150c is inserted through the uppermost opening in the partition insert 126 as shown in FIG. 21, and it is likewise locked into place. An endoscope may be inserted into the lowermost opening of the insert 126 and used to observe the procedure performed through the access port 114.

FIG. 22 illustrates that the proximal-end curvature of the tubes 150a-c is preferably such that the tubes will angle away from one another in the sections lying proximal to the access port 114. This minimizes interference between the handles of instruments inserted through the tubes 150a-c.

The access ports and tubes may be used to implant a gastric band (e.g. Lap-Band or Swedish Band) using methods similar to those disclosed in U.S. application Ser. No. ______, filed Sep. 12, 2008, Attorney Docket No. TRX-1110, with either one of the disclosed access devices alone or in combination with the tubes 150a-c being used (in place of the cannula and access device described in that application) to give access to the snare, dissection instrument etc. As discussed previously, where the access device 10a of FIG. 2 is to be used to implant the gastric band, the band may be dropped through the lumen 18a in the base 12a and into the operative space before the seal 14a is coupled to the base 12a.

It should be recognized that a number of variations of the above-identified embodiments will be obvious to one of ordinary skill in the art in view of the foregoing description. Accordingly, the invention is not to be limited by those specific embodiments and methods of the present invention shown and described herein. Rather, the scope of the invention is to be defined by the claims and their equivalents.

Any and all applications referred to herein, including for purposes of priority, are hereby incorporated herein by reference.

We claim:

1. A surgical access device, comprising:
a base including a circumferential wall defining a lumen;
a seal on a proximal portion of the base, the seal including a proximal surface and a plurality of tubular instrument ports extending proximally from the proximal surface, the ports pivotable relative to the wall.

2. The access device of claim 1, wherein the longitudinal axes of the ports are parallel to each other.

3. The access device of claim 2, wherein the ports are parallel to the longitudinal axis of the lumen.

4. The access device of claim 1, where the base includes a lower flange surrounding a distal opening in the base.

5. The access device of claim 4, wherein the flange includes an inner portion and an outer portion positioned radially outwardly from the inner portion, and wherein the outer portion extends proximally of the inner portion.

6. The access device of claim 5, wherein the outer portion of the flange is deflectable relative to the inner portion.

7. The access device of claim 4, where the flange has a concave proximal surface.

8. The access device of claim 1, wherein the proximal surface of the seal includes a convex portion.

9. The access device of claim 1, wherein the proximal surface of the seal is a convex surface.

10. The access device of claim 8, wherein the plurality of ports extend from the convex portion of the proximal surface.

11. The access device of claim 10, wherein at least one of the ports forms a junction with the convex portion, the junction having a first region defining a curve and a second region defining an apex.

12. The access device of claim 10 wherein the junction has an approximate teardrop shape.

13. The access device of claim 1, further including a plurality of clips detachably coupling the seal to the base.

14. The access device of claim 1, wherein the seal is attached to the base.

15. The access device of claim 1, wherein the seal includes a plurality of openings, and wherein the ports are detachable coupled to the openings in the seal.

16. The access device of claim 1, further including a plurality of caps, each cap having a lumen and a seal within the lumen, the caps attachable to corresponding ones of the ports.

17. The access device of claim 1 wherein each port has a port lumen and a seal in the port lumen, wherein a first plurality of the port lumens has a first minimum diameter, and a second plurality of the port lumens has a second minimum diameter smaller than the minimum diameter of the first plurality of port lumens.

18. The access device of claim 1, further including a plurality of instrument tubes having distal portions slidable through the ports and through the lumen of the base, each instrument tube including an instrument lumen for receiving a medical instrument.

19. The access device of claim 18, wherein at least one of the instrument tubes includes an elongate shaft having a curved distal portion.

20. The access device of claim 19, wherein the distal portion of the elongate shaft has a preformed curve.

21. The access device of claim 20, wherein the curved distal portion includes a deflectable section and wherein the instrument tube includes an actuator engageable to deflect the deflectable section.

22. The access device of claim 20, wherein the deflectable section is flexible and wherein the instrument tube includes a rigid proximal section.

23. The access device of claim 1, wherein at least one of the ports includes a first seal comprising a self-sealing seal positioned to seal the port lumen when no instruments are positioned within the port lumen, and a second seal positioned to seal against shafts of instruments positioned within the port lumen.

24. The access device of claim 1, further including an insufflation port fluidly coupled to the base.

25. A surgical access system, including:
an access port positionable in an incision in body tissue; and
at least three instrument tubes insertable through the port into a body cavity, each tube having an instrument lumen and a precurved distal end.

26. The surgical access system of claim 25, wherein the instrument tubes are oriented in the access port such that the curvature of each distal end directs instruments passed the instrument tubes towards a common treatment site in the body cavity.

27. The surgical access system of claim 26, wherein the curved distal portion of at least one of the instrument tubes includes a deflectable section and wherein the instrument tube includes an actuator engageable to deflect the deflectable section.

28. A method of gaining access to a body cavity, the method comprising:
providing an access device including a base having a circumferential wall defining a lumen, a seal on a proximal portion of the base, and a plurality of tubular instrument ports extending proximally from a proximal surface of the seal;
forming a percutaneous incision in the body;
positioning the access port with the incision, with the base disposed within the incision and the seal external to the incision;
inserting at least two instruments into corresponding ones of the ports, and performing a procedure using the instruments.

29. The method according to claim 1, wherein the method further includes causing at least one of the ports to pivot relative to the proximal surface of the seal in response to manipulation of the instrument in the at least one port.

30. The method according to claim 28, wherein positioning the access device includes positioning the base within the incision with the circumferential wall and then coupling the seal to the base.

31. The method according to claim 30, further including, prior to coupling the seal to the base, passing an implant device through the lumen of the base into the body cavity.

32. The method according to claim 28, wherein inserting at least two instruments includes inserting an instrument tube through one of the ports, and passing an instrument through the instrument tube into the body cavity.

33. The method according to claim 32, wherein inserting at least two instruments includes inserting a second tube through a second one of the ports and passing a second instrument through the second instrument tube into the body cavity.

34. The method according to claim 33 wherein the first and second instrument tubes are provided to have curved distal ends, and wherein the method includes adjusting the rotational orientation of each instrument tube such that the curvature of each distal end directs the first and second instruments towards a common treatment site in the body cavity.

35. The method according to claim 34, further including the step of deflecting the distal end of at least one of the instrument tubes within the body cavity.

36. The method according to claim 35, including the step of passing an endoscope through a third one of the instrument ports and observing the procedure using the endoscope.
